(19) United States
(12) Patent Application Publication
(10) Pub. No.: US 2025/0161247 A1
(43) Pub. Date: May 22, 2025

(54) METHOD FOR TREATING BREAST CANCERS AND PARP RESISTANT BREAST CANCERS

(71) Applicant: LANTERN PHARMA INC., Dallas, TX (US)

(72) Inventors: Aditya KULKARNI, Dallas, TX (US); Kishor BHATIA, Dallas, TX (US)

(73) Assignee: LANTERN PHARMA INC., Dallas, TX (US)

(21) Appl. No.: 19/023,098

(22) Filed: Jan. 15, 2025

Related U.S. Application Data
(63) Continuation of application No. PCT/US2023/070328, filed on Jul. 17, 2023.
(60) Provisional application No. 63/368,510, filed on Jul. 15, 2022.

(51) Int. Cl. A61K 31/17; A61K 31/122; A61K 31/337; A61K 31/454; A61K 31/502; A61K 31/55; A61K 33/243; A61P 35/00

(52) U.S. Cl. CPC A61K 31/17; A61K 31/122; A61K 31/337; A61K 31/454; A61K 31/502; A61K 31/55; A61K 33/243; A61P 35/00

(57) ABSTRACT

A method of treating breast cancer includes a combination of a therapeutically effective amount of an illudin or an illudin analog thereof, derivative, or a pharmaceutically acceptable salt thereof; and a therapeutically effective amount of a PARP inhibitor. Compositions and kits of the same are included herein. The breast cancer may be refractory to various PARP inhibitors.

### CROSS-REFERENCE TO RELATED APPLICATION

This application is a continuation of International Application No. PCT/US2023/070328 Filed Jul. 17, 2023, which claims priority to and the benefit of U.S. Provisional Patent Application No. 63/368,510, filed Jul. 15, 2022, which are incorporated by reference herein in their entireties.

### TECHNICAL FIELD

This application relates to treatments of breast cancer. More particularly, this application relates to a combination treatment of breast cancers that are refractory or in relapsed subjects and that were previously treated with a PARP inhibitor. Further, the combination treatment can be used to treat triple negative breast cancers.

### BACKGROUND

Breast cancer is the most common female cancer worldwide and the second most common cause of cancer-related deaths in women in the United States. Triple-negative breast cancer (TNBC) is a subtype of breast cancer that is characterized by the deficiency of estrogen receptor (ER), progesterone receptor, and human epidermal growth factor receptor 2 (HER2). While tremendous advances have been made in other subtypes of breast cancer such as HER2⁺ tumors with the development of targeted agents against the HER2 receptor and ER⁺ breast cancers with agents to block estrogen signaling, there are no approved or effective targeted treatments for TNBC. Historically, cytotoxic chemotherapy has been the only viable systemic treatment option for patients with TNBC.

Triple-negative breast cancer accounts for about 10-20% of all breast cancer cases. It tends to occur more frequently in younger women and those with certain genetic mutations, such as BRCA1. TNBC often presents as a more aggressive form of breast cancer, with a higher likelihood of recurrence and metastasis (spread to other organs) compared to other breast cancer subtypes. Due to its lack of hormone receptors and HER2 overexpression, treatment options for TNBC are limited to chemotherapy, surgery, and radiation therapy.

Accordingly, there is always a need for an improved treatment for breast cancer. It is to this need, among others, that this application is directed.

### SUMMARY

This application discloses the discovery of a treatment for breast cancer in subjects using a combination of an illudin or illudin analog (e.g., acylfulvene) and PARP inhibitor(s). Such a treatment can have greater effects than the effects provided by either acylfulvene or PA inhibitor treatment(s), alone.

One aspect of this application includes a combination therapy for treating cancers. In embodiments, the therapy includes administering a combination of active agents including an illudin or illudin analog acylfulvene), and a PARP inhibitor. The methods include administering to a subject in need of treatment a combination of active agents having a therapeutically effective amount of an illudin or an illudin analog thereof, derivative, or a pharmaceutically acceptable salt thereof; and a therapeutically effective amount of a PARP inhibitor, or a pharmaceutically acceptable salt thereof. The subject can have relapsed cancer and/or refractory cancer. The subject may have been treated previously with a PARP inhibitor. The subject may have been treated with the combination after the cancer is refractory and resistant to a PARP inhibitor.

One aspect includes a method in which the subject subsequently relapsed more than about 1 month, 2 months, 3 months, 4 months, or more following the cessation of treatment with the PARP inhibitor.

Another aspect includes a method in which the illudin analog is an acylfulvene.

Another aspect includes a method in which the illudin analog is HydroxyUreaMethylAcylfulvene.

Another aspect includes a method in which the illudin analog has the following structure:

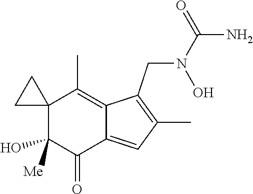 [SMILES: CC1=C(CN(O)C(N)=O)C2=C(C)C3(CC3)[C@@](C)(O)C(=O)C2=C1]

Another aspect includes a method in which the illudin analog has the following structure:

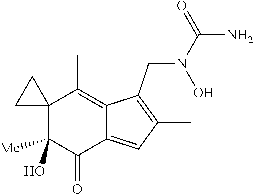 [SMILES: CC1=C(CN(O)C(N)=O)C2=C(C)C3(CC3)[C@](C)(O)C(=O)C2=C1]

Another aspect includes a method in which the illudin analog is Irofulven.

Another aspect of this application provides pharmaceutical compositions comprising an illudin or illudin analog (e.g., acylfulvene) and a PARP inhibitor or pharmaceutically acceptable salts thereof, mixed with pharmaceutically suitable carriers or excipient(s) at doses to treat or prevent cancer, primarily those who have been previously treated with a PARP inhibitor. The pharmaceutical compositions can also be administered in combination with other therapeutic agents or therapeutic modalities simultaneously, sequentially, or in alternation.

Another aspect of this application includes the therapy including an acylfulvene that is (−)-hydroxyureamethyl acylfulvene

### BRIEF DESCRIPTION OF THE DRAWINGS

FIG. 1 shows nanomolar concentrations of LP-184 inhibited in vitro viability of 6 NCI-60 breast cancer cell lines.

FIG. 2 shows HBCx10 tumor volume in a PDX HBCx10 model over time during a treatment with LP-184 and/or Olaparib in HRD positive PARPi resistant tumors.

FIG. 3 shows HBCx10 tumor volume in a PDX HBCx10 model over time during a treatment with LP-184 and/or Olaparib in HRD positive PARPi sensitive tumors.

FIG. 4 shows tumor volume in a PDX HBCx10 model over time during a treatment with LP-184 and Olaparib

FIG. 5 shows tumor volume in a PDX HBCx28 model over time during a treatment with LP-184 and Olaparib.

FIG. 6 shows complete tumor regression in an HR deficient, multi-drag resistant TNBC in PDX HBCx-24 model.

### DETAILED DESCRIPTION

This application provides therapeutic methods to treat breast cancer in a subject. One embodiment includes a method for treating persons with breast cancers and breast cancers resistant to PARP inhibitors in which that method include s administration of an effective amount of acylfulvene (e.g., hydroxyureamethyl acylfulvene) or salt thereof and a PARP inhibitor.

### Illudin or Acylfulvene

In one embodiment, this application includes the use of an illudin or illudin analog (e.g., acylfulvene). Acylfulvene (also known as 6-Acetylfulvene) is a synthetic anticancer drug that belongs to the class of alkylating agents. It was derived from a natural product called fulvic acid, which is found in plants and soil. Acylfulvene is a class of cytotoxic semi-synthetic derivatives of illudin, a natural product that can be extracted from the jack o'lantern mushroom (Omphalotus olearius). Acylfulvene, derived from the sesquiterpene illudin S by treatment with acid (reverse Prins reaction), is far less reactive to thiols than illudin S. Acylfulvene works by alkylating DNA, which means it forms covalent bonds with the DNA molecules in cells, leading to damage and interfering with the replication and transcription processes. This action prevents cancer cells from dividing and ultimately induces cell death.

In one example, the acylfulvene is (−)-hydroxyureamethyl acylfulvene (termed LP-184 by Lantern Pharma Inc.), which shifts light negatively, is shown below:

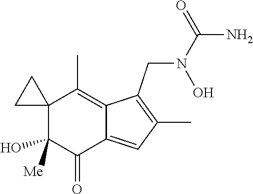 [SMILES: CC1=C(CN(O)C(N)=O)C2=C(C)C3(CC3)[C@@](C)(O)C(=O)C2=C1]

In another example, the acylfulvene is (+)-hydroxyureamethyl acylfulvene (termed LP-284 by Lantern Pharma Inc.), which shifts light positively, is shown below:

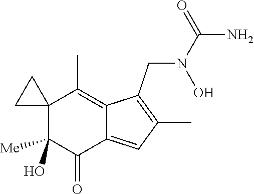 [SMILES: CC1=C(CN(O)C(N)=O)C2=C(C)C3(CC3)[C@](C)(O)C(=O)C2=C1]

(+)-hydroxyureamethyl acylfulvene and (−)-hydroxyureamethyl acylfulvene are enantiomers and are now known publicly.

In another example, the acylfulvene is Irofulven.

LP-284 and LP-184 are enantiomers and are now known publicly.

In another example, the acylfulvene is Irofulven or 6-hydroxymethylacylfulvene.

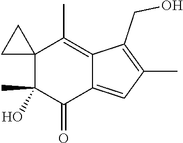 [SMILES: CC1=C(CO)C2=C(C)C3(CC3)[C@@](C)(O)C(=O)C2=C1]

### PARP Inhibitor

PARP inhibitors are & type of cancer drug. PARP stands for poly adenosine diphosphate-ribose polymerase (poly-ADP ribose polymerase (PARP) inhibitor), a type of enzyme that helps repair DNA damage in cells. PARP inhibitors work by preventing cancer cells from repairing damaged DNA, allowing them to die. PARP enzymes help repair DNA damage. Blocking them can keep cancer cells from repairing, and this allows them to die. There are at least four main PARP inhibitor olaparib (Lynparza), niraparib (Zejula), rucaparib (Rubraca) and talazoparib (Talzenna). Pharmaceutical compositions are disclosed that include one or more PARP inhibitor and typically at least one additional substance, such as an excipient, a known therapeutic other than those of the present disclosure, and combinations thereof. In some embodiments, a PARP used or can in combination with other agents known to have beneficial, additive or synergistic activity with the PARP inhibitor. In one specific embodiment, the PARP inhibitor is a PARP-1 inhibitor. In other embodiments, the PARP inhibitor is an inhibitor of any enzyme of the PARP family, e.g., PARP1 and/or PARP2.

Examples of suitable PARP inhibitors according to the invention include, but are not limited to, olaparib (AZD-2281, 4-[(3-[(4-cyclopropylcarbonyl)piperazin-4-yl]carbonyl)-4-fluorophenyl]met-hyl(2H)-phthalazin-1-one), veliparib ABT-888, CAS 912444-00.9, 2-((fi)-2-methylpyrrolidin-2-yl)-1W-benzimidazole-4-carboxamide), CEP-8983 (II-methoxy-4,5,6,7-tetrahydro-1H-cyclopenta[a]pyrrolo[3,4-c]car-bazole-1,3 (2H)-dione) or a prodrug thereof (e.g. CEP-9722), rucaparib (AG014699, PF-01367338, 8-Fluoro-2-{4-[(methylamino)methyl]phenyl}-1,3,4,5-tetrahydro-6H-azepino[−5,4,3-cd]indol-6-one), E7016 (GPI-21016, 10-((4-Hydroxypiperidin-1-yl)methyl)chromeno-[4,3,2-de]phthalazin-3(2H)-o-ne), talazoparib (BMN-673, (8S,9R)-5-fluoro-8-(4-fluorophenyl)-9-(1-methyl-1H-1,2,4-triazol-5-yl)-8-9-dihydro-2H-pyrido[4,3,2de]phthalazin-3 (7H)-one), INO-1001 (4-phenoxy-3-pyrrolidin-1-yl-5-sulfamoyl-benzoic acid), KU0058684 (CAS 623578-11-0), nirapanib (MK 4827, Merck & Co Inc), iniparib (BSI 201), iniparib-met (C-nitroso metabolite of Iniparib), CEP 9722 (Cephalon Inc), LT-673, MP-124, NMS-P118, XAV939, AZD 2461, nicotinamides, 5-methyl nicotinamide, 4-Amino-1,8-naphthalimide, picolinamide, benzamides, 3-substituted benzamides, 3-methoxybenzamide, 3-hydroxybenzamide, 3-aminobenzamide, 3-chloroprocainamide, 3 nitrosobenzamide, 4 aminobenzamide, 2-aminobenzamide, methyl 3,5-diiodo-4-(4′-methoxyphenoxy)benzoate, methyl-3,5-diiodo-4-(4′-methoxy-3′,5′-diiodo-phenoxy)benzo cyclic benzamides, 1,5-di[(3-carbamoylphenyl)aminocarbonyloxy]pentane, indoles, benzimidazoles, benzoxazole-4-carboxamides, benzimidazole-4-carboxamides, 2-substituted benzoxazole 4-carboxamides, 2-substituted benzimidazole 4-carboxamides, 2-aryl benzimidazole 4-carboxamides, 2-cycloalkylbenzimidazole-4-carboxamides 2-(4-hydroxphenyl)benzimidazole, A-carboxamide, quinoxalinecarboxamides, imidazopyridinecarboxamides, 2-phenylindoles, 2-substituted benzoxazoles, 2-phenyl benzoxazole, 2-(3-methoxyphenyl)benzoxazole, 2-substituted benzimidazoles, 2-phenyl benzimidazole, 2-(3-methoxyphenyl)benzimidazole, 1,3,4,5-tetrahydro-azepino[5,4,3-cd]indol-6-one, azepinoindoles, azepinoindolones, 1,5-dihydro-azepino[4,5,6-cd]indolin-6-one, dihydrodiazapinoindolinone, 3-substituted dihydrodiacapinoindolinones, 3-(4 trifluoromethylphenyl)-dihydrodiazapinoindolinone, tetrahydrodiazapinoindolinone, 5,6-dihydroimidazo[4,5,1-j,k][1,4]benzodiazopin-7 (4H)-one, 2-phenyl-5,6-dihydro-imidazo[4,5,1-jk][1,4]benzodiazepin-7(4H)-one, (2,3-dihydro-isoindol-1-one, benzimidazole-2-piperazine, benzimidazole-2-piperazine heterocyclic derivatives, 4-iodo-3-nitrobenzamide, benzopyrones, 1,2-benzopyrone 6-nitrosobenzopyrone, 6-nitroso 1,2-benzopyrone, 5-iodo-6-aminobenzopyrone, benzoylurea, quinolone, isoquinolone, isoquinolinones, dihydroisoquinolinones, 2H-isoquinolin-1-ones, 3H-quinazolin-4-ones, 5-substituted dihydroisoquinolinones, 5-hydroxy dihydroisoquinolinone, 5-methyl dihydroisoquinolinone, 5-hydroxy isoquinolinone, 5-amino isoquinolin-1-one, 5-dihydroxyisoquinolinone, 1,5-dihydroxyisoquinoline, 1,5-isoquinolinediol, 4-hydroxyquinazoline, substituted thiazolyl-isoquinolinones, substituted oxazoyl-isoquinolinones, tetrahydro-2H-isoquinolin-1-one, 3,4-dihydroisoquinolin-1(2H)-ones, 3,4-dihydro-5-methoxy-isoquinolin-1(2H)-one, 3,4-dihydro-5-methyl-1(2H)isoquinolinone, 3H-quinazolin 4-one, isoquinolin-1(2H)-ones, 3,4 dihydroisoquinolin-1(2H)-one, 4-carboxamido-benzimidazole, substituted 6-cyclohexylalkyl substituted 2-quinolinones, substituted 6-cyclohexylalkyl substituted 2-quinoxalinones, 7-phenylalkyl substituted 2-quinolinones, 7-phenylalkyl substituted 2-quinoxalinones, 6-substituted 2-quinolinones, 6-substituted 2-quinoxalinones, 1-(arylmethyl)quinazoline-2,4(1H,3H)-dione, 4,5-dihydro-imidazo[4,5,1-ij]quinolin-6-ones, 1,6-naphthyridine-5(6H)-ones, 1,8-naphthalimides, 4-amino-1,8-naphthalimides, 3,4-dihydro-5-[4-1(1-piperidinyl)butoxy]-1(2H)-isoquinolinone, 2,3-dihydrobenzo[de]isoquinolin-1-one, 1-1 lb-dihydro-[2H]benzopyrano[4,3,2-de]isoquinolin-3-one, tetracyclic lactams, benzpyranoisoquinolinones, benzopyrano[4,3,2-de]isoquinolinone, quinazolines, quinazolinones, quinazolinediones, A-hydroxyquinazoline, 2-substituted quinazolines, 8-hydroxy-2-methylquinazolin-4-(3H)one, phthalazines, phthalazinones, phthalazin-1(2H)-ones, 5-methoxy-4-methyl-1(2) phtbalazinones, 4-substituted phthalazinones, 4-(1-piperazinyl)-1(2H)-phthalazinone, tetracyclic benzopyrano[4,3,2-de]phthalazinones and tetracyclic indeno[1,2,3-de]phthalazinones, tricyclic phthalazinones, 2-aminophthalhydrazide, phthalazinone ketone, dihydropyridophthalazinone, 6-substituted 5-arylamino-1 h-pyridine-2-ones, pyridazinones, tetrahydropyridopyridazinone, tetraaza phenalen-3-one, thieno[2,3-c]isoquinolin-5-one (TIQ-A), 2,5-diazabicyclo[2.2.1]heptane, pyrimidoimidazole, isoindolinones, phenanthridines, phenanthridinones, 5[H]phenanthridin-6-one, substituted 5[H]phenanthridin-6-ones, 2,3-substituted 5[H]phenanthridin-6-ones, sulfonamide/carbamide derivatives of 6(5H)phenanthridinones, thieno[2,3-c]isoquinolones, 9-amino thieno[2,3-c]isoquinolone, 9-hydroxythieno[2,3-c]isoquinolone, 9-methoxythieno[2,3-c]isoquinolone, N-(6-oxo-5,6-dihydrophenanthridin-2-yl]-2-(N,N-dimethylamino)acetamide, substituted 4,9-dihydrocyclopenta[imn]phenanthridine-5-ones, unsaturated hydroximic acid derivatives, O-(3-piperidino-2-hydroxy-1-propyl)nicotinic amidoxime, O-(2-hydroxy-3-piperidino-propyl)-3-carboxylic acid amidoxime, pyridazines, pyrazinamide, BGB-290, PF-1367338 (Pfizer Inc), AG014600 (Pfizer, Inc.), KU-59436 (KuDOS/AstraZeneca PJ34, 4-amino-1,8-naphthalimide (Trevigen), 6(5H)-phenanthridinone (Trevigen), NU1025, 4-HQN, BGP-15, A-966492, GPI21016, 6(5H)-phenanthridinone (Phen), theobromine, theophylline, caffeine, methylxanthines, thymidine, 3-aminophtalhydrazide, analogs, derivatives or a mixture thereof.

Additional PARP inhibitors are described for example in WO14201972, WO14201972, WO12141990, WO10091140, WO9524379; WO09155402, WO009046205 WO08146035. WO08015429 WO0191796, WO0042040, US2006004028, EP2604610, EP1802578, CN104140426, CN104003979, US060229351, U.S. Pat. No. 7,041,675, WO07041357, WO2003057699, U.S. Ser. No. 06/444,676, US20060229289, US20060063926, WO2006033006, WO2006033007, WO03051879, WO2004108723, WO2006066172, WO2006078503, US20070032489, WO2005023246, WO2005097750, WO2005123687, WO2005097750, U.S. Pat. Nos. 7,087,637, 6,903,101, WO20070011962, US20070015814, WO2006135873, UA20070072912 WO2006065392, WO2005012305, WO2005012305, EP412848, EP453210, EP454831, EP879820, EP879820, WO030805, WO03007959, U.S. Pat. No. 6,989,388, US20060094746, EP1212328, WO2006078711, U.S. Ser. No. 06/426,415, U.S. Ser. No. 06/514,983, EP12 US20040254372, US20050148575, US20060003987, U.S. Ser. No. 06/635,642, WO200116137, WO2004105700, WO03057145A2, WO2006078711, WO2002044157, US20056924284, WO2005112935, US20046828319, WO2005054201, WO2005054209, WO2005054210, WO2005058843, WO2006003146, WO2006003147, WO2006003148, WO2006003150, WO2006003146, WO2006003147, UA20070072842, U.S. Ser. No. 05/587,384, US20060094743, WO2002094790, WO2004048339, EP1582520, US20060004028, WO2005108400, U.S. Pat. No. 6,964,960, WO20050080096, WO2006137510, UA20070072841, WO2004087713, WO2006046035, WO2006008119, WO06008118, WO2006042638, US2006022928, US20060229351, WO2005023800, WO1991007404, WO2000042025, WO2004096779, U.S. Pat. No. 6,426,415 WO2068407, U.S. Pat. No. 6,476,048, WO2001090077, WO2001085687, WO2001085686, WO2001079184, WO2001057038, WO2001023390, WO01021615A1, WO2001016136, WO2001012199, WO95024379, WO200236576, WO2004080976, WO2007149451, WO2006110816, WO2007113596, WO2007138351, WO2007144652, WO2007144639, WO2007138351, WO2007144637, Banasik et al. (J. Biol. Chem., 267:3 1:5, Banasik et al. (Molec. Cell. Biochem, 138:185-97, 1994), Cosi et al. (Expert Opin, Ther. Patents 12 (7), 2002), Southan and Szabo (Curr Med Chem, 10 321-340, 2003), Underhill C. et al. (Annals of Oncology, doi:10.1093/annonc/mdq322, pp 1-12, 2010), Murai J. et al. (J. Pharmacol. Exp. Ther., 349:408-416, 2014), all these patents and publications being hereby incorporated by reference in their entirety.

In a preferred embodiment, the PARP inhibitor compound is selected from the group consisting of rucaparib (AG014699, PF-01367338) olaparib (AZD2281), veliparib (ABT888), iniparib (BSI 201), niraparib (MK 4827), talazoparib (BMN673), AZD 2461, CEP 9722, E7016, INO-1001, LT-673, MP-124, NMS-P118, XAV939, analogs, derivatives or a mixture thereof.

In an even more preferred embodiment, the PARP inhibitor is selected from the group consisting of rucaparib. Olaparib, veliparib, iniparib, miraparib, talazoparib, AZD 2461, analogs, derivatives or a mixture thereof.

In one embodiment, acylfulvene or hydroxyureamethyl acylfulvene or its salt may be administered either prior to concomitantly with, or subsequent to the a ministration of a PARP inhibitor.

One aspect of this application includes a method of treating cancer in a subject in need thereof. The method involves administering to the subject an effective amount of a PARP inhibitor an effective amount of an acylfulvene. A PARP inhibitor may be administered prior to or concomitantly with an acylfulvene for optimal synergistic effects.

Another embodiment includes a pharmaceutical composition having a therapeutically effective amount of an illudin or an illudin analog thereof, derivative, or a pharmaceutically acceptable salt thereof, and a therapeutically effective amount of a PARP inhibitor or an anal re, or a pharmaceutically acceptable salt thereof. The illudin analog can be HydroxyUreaMethylAcylfulvene.

In another embodiment, a kit for the treatment of cancer in a subject includes a therapeutically effective amount of an illudin or an illudin analog thereof, derivative, or a pharmaceutically ac able salt thereof, and a therapeutically effective amount of a PARP inhibitor or an analog, derivative, or a pharmaceutically acceptable salt thereof:

In one embodiment, the breast cancer can be treated with the following regimens: Histologically confirmed breast cancer, PARP resistant/refractory disease, can be defined as disease progression within 180 days following the last administered dose of PARP therapy (resistant), or lack of response or disease progression while receiving the most recent PARP based therapy (refractory), respectively.

PARP inhibition causes synthetic lethality in breast cancers associated with germline BRCA and BRCA mutations and is routinely used in clinical practice for metastatic breast cancer. Breast cancers with homologous recombination deficiency or BRCAness, most commonly triple-negative breast cancers, may also benefit. Currently ARP inhibitor use for triple-negative breast cancer with wild-type BRCA does not have definitive efficacy. One embodiment includes treating breast cancer by administering an effective amount of cancer acylfulvene or hydroxyureamethyl acylfulvene or its salt.

In one example, the tumor cells are NERD or NER tumors. NER deficient cancers refer to a group of cancers related to Nucleotide Excision Repair (NER) pathway deficiencies. When there are defects or mutations in the NER pathway, it can lead to the accumulation of DNA damage, which may increase the risk of developing certain types of cancers. NER is a cellular mechanism responsible for repairing DNA damage caused by environmental fact such as UV radiation and certain chemicals. NERD or NER deficient tumors are nucleotide excision repair deficient tumors whose phenotype is a result of mutations in genes responsible for excision DNA repair—these include but are not limited to—ERCC1, ERCC3, ERCC4, ERCC5, ERCC6, RAD50, ATR, ATM, MRE, CSB, XPD and others

In one embodiment, the patient is treated with radiation prior to a treatment with an acylfulvene.

In another embodiment, a second therapeutic can be one or more chemotherapeutic agents selected from camptothecin derivatives, paclitaxel, docetaxel, epothilone B, 5-FU, gemcitabine, oxaliplatin, cisplatinum, carboplatin, melphalan, dacarbazine, temozolomide, doxorubicin, imatinib, erlotinib, bevacizumab, cetuximab and a Raf kinase inhibitor.

In another embodiment, the second therapeutic is one or more chemotherapeutic agents selected from paclitaxel or cisplatinum.

Therapy may be “first-line”, i.e., as an initial treatment in patients who have had no prior anti-cancer treatments, either alone or in combination with other treatments; of “second-line”, as a treatment in patients who have had one prior anti-cancer treatment regimen, either alone or in combination with other treatments; or as “third-line”, “fourth-line”, etc. treatments, either alone or in combination with other treatments.

In another embodiment, a kit for the treatment of cancer in a subject includes a therapeutically effective amount of an illudin or an illudin analog thereof, derivative, or a pharmaceutically acceptable salt thereof, and a therapeutically effective amount of a PARP inhibitor or an analog, derivative, or a pharmaceutically acceptable thereof.

In another embodiment, the second therapeutic is one or more chemotherapeutic agents selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, cetuximab and a Raf kinase inhibitor.

In another embodiment, the second therapeutic is one or more chemotherapeutic agents selected from paclitaxel or cisplatinum.

The term “combination therapy” can include or includes the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of therapeutic agents and non-drug treatment is achieved. For example, in appropriate cases, the beneficial effect is still ach e non-drug treatment is temporally removed from the administration of the therapeutic agents, perhaps by days or even weeks or months.

In another aspect, a composition or combination therapy herein, or a pharmaceutically acceptable salt or solvate thereof, may be administered in combination with radiation therapy. Radiation therapy can also be administered in combination with a composition of the present invention and another chemotherapeutic agent described herein as part of a multiple agent therapy.

Combination therapy can be achieved by administering two or more acylfulvene, a PARP inhibitor and one or more other therapeutic agents, each of which is formulated and administered separately, or by administering two or more agents in a sing formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present within the patient's body at the same time, this need not be so.

The methods of combination therapy may or should result in a synergistic effect, wherein the effect combination of compounds or other therapeutic age s is greater than the sum of the effects resulting from administration of any of the compounds or other therapeutic agents as single agents. A synergistic effect may also be an effect that cannot be achieved by administration of any of the compounds or other therapeutic agents as a single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

Therapeutically effective doses can vary, as recognized by those skilled in the art, depending on the diseases treated, the severity of the disease, the route of administration, the age and general health condition of the patient, excipient usage, the possibility of co-usage with other therapeutic treatments such as use of other agents and the judgment of the treating physician For example, guidance for selecting an effective dose can be determined by reference to the prescribing information for acylfulvene or hydroxyureamethyl acylfulvene or journal discussion the same.

The term “effective amount” as used herein refers to the amount of an agent needed to alleviate at least one or more symptoms of the disease or disorder, and relates to a sufficient amount of pharmacological composition to provide the desired effect. The term “therapeutically effective amount” therefore refers to an amount of the agent that is sufficient to provide a particular effect when administered to a typical subject. An effective amount may be an amount sufficient to decrease the symptoms of a disease responsive to inhibition of a PARP inhibitor. For cancer therapy, efficacy in vivo can, for example, be measured by assessing the duration of survival, time to dis progression (TTP), the response rates (RR), duration of response, and/or quality of life. Effective amounts may vary, as recognized by those skilled in the art, depending on route of administration, excipient usage, and co-usage with of ter agents. An effective amount as used herein, in various contexts, wot also include an amount sufficient to delay the development of a symptom of the disease, al the course of a symptom disease (for example but not limited to, slowing the progression of a symptom of the disease), or reverse a symptom of the disease. Thus, it is not generally practicable to specify an exact “effective amount”. However, for any give case, an appropriate “effective amount” can be determined by one of ordinary skill in the art using only routine experimentation.

The dosage ranges for the administration of an agent according to the methods described herein depend upon, for example, the form of the its potency, and the extent to which symptoms, markers, or indicators of a condition described herein are desired to be reduced, for example, the percentage reduction desired for tumor growth. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of an agent described herein in, e.g., the treatment of a condition described herein, or to induce a response as described herein (e.g., solid cancers or blood cancers) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% follow treatment according to the methods described herein. Efficacy can be assessed, for example, by measuring a marker, indicator, symptom, and/or the incidence of a condition treated according to the methods described herein or any of her measurable parameter appropriate, e.g. tumor size and/or growth rate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes. (1) inhibiting the disease, e.g., preventing a worsening of symptoms (e pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for exam e, treatment of blood cancers in a mouse model. When using an experimental animal model, efficacy of treatment is evidenced when a statistically significant change in a marker is observed, e.g. tumor size and/or growth rate. In some embodiments, the therapeutically effective amount of hydroxyureamethyl-acylfulvene, acylfulvene, Irofulven or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day, 580 mg/day, 600 mg/day, 620 mg/day, 640 mg/day 680 mg/day, and 720 mg/day.

The administration dose should be adjusted for the requirement of the individual in need. The dosing of PARP (Poly ADP-ribose polymerase) inhibitors can vary depending on the specific drug being used and the medical condition it's prescribed for. PARP inhibitors are a type of targeted therapy used in the treatment of certain types of cancers, especially those with in DNA repair mechanisms. For illustration, Olaparib (Lynparza). For ovarian cancer, the typical dose is 300 mg twice daily (total daily dose of 600 mg). For breast cancer, it is 300 mg twice daily (total daily dose of 600 mg) for those with germline BRCA mutations. Further, Niraparib (Zejula) The typical dose is 300 mg once daily for maintenance of ovarian cancer. The dose may be adjusted based on individual patient factors. Further Rucaparib (Rubraca): The typical dose is 600 mg twice daily (total daily dose of 1200 mg) for ovarian cancer and other related indications. The actual dose prescribed to a patient can be determined by their healthcare provider based on various factors, including the specific cancer being treated, the patient's overall health, and other individual considerations.

The term “treat” is used and includes both therapeutic treatment and prophylactic treatment (reducing the likelihood of development). Both terms mean decrease, suppress, attenuate, diminish, arrest, or stabilize the development or progression of disease (e.g., a disease or disorder delineated herein), lessen the severity of the disease or improve the symptoms associated with the disease.

The pharmaceutical compositions can be included in a container, pack, or dispenser together with instructions for administration.

The composition of the present invention is capable of further forming salts. The composition of the present invention can form more than one salt per molecule, e.g., mono-, di-, tri-. All of these forms are also contemplated within the scope of the claimed invention.

As used herein, “pharmaceutically acceptable salts” refer to derivatives of the compounds of the present invention wherein ent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition from forms (solvates), of the same salt.

As used herein, the term “selectively” means tending to occur at a higher frequency in one population than in another population. The compared populations can be cell populations. Preferably, an event occurs selectively in population A relative to population B if it occurs greater than two times more frequently in population A as compared to population B. An event occurs if it occurs greater than five times more frequently in population A. An event occurs selectively if it occurs greater than ten times more frequently in population A; more preferably, greater than fifty times; even more preferably, greater than 100 times; and most preferably, eater than 1000 times more frequently in population A as compared to population B. For example, cell death would be said to occur selectively in cancer cells if it occurred greater than twice as frequently in cancer cells as compared to normal cells.

The composition, or pharmaceutically acceptable salts or solvates thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous of organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the invention. The examples do not limit the claimed invention. Based on the present disclosure the skilled artisan can identify and employ other components and methodology useful for practicing the present invention.

As used herein, a “subject in need thereof” is a subject having a precancerous condition. Preferably a subject in need thereof has cancer. A “subject” includes a mammal. The mammal can be e.g., any mammal, e.g., a human, primate, bird, mouse, rat, dog, cat, cow, horse, goat sheep or a pig. Preferably, the mammal is a human. The subject of the present invention includes any human subject who has been diagnosed with, has symptoms of, or is at risk of developing a cancer or a precancerous condition.

A subject in need thereof may have refractory resistant cancer. “Refractory or resistant cancer” means that not respond to treatment. The cancer may resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments the prior therapy is monotherapy. In certain embodiments the prior therapy is combination therapy.

A “relapsed cancer” is a cancer that has previously been treated and, as a result of that treatment, the subject made a complete or partial recovery (i.e. the subject is said to be in remission), but that after the cessation of the treatment the cancer returned or worsened. Put another way, a relapsed cancer is one that has become resistant to a treatment, after a period in which it was effective and the subject made a complete or partial recovery. Typically, in patients with breast cancer, the development of resistance and refractory disease occurs after multiple rounds of treatment. Further, PARP inhibitor can become refractory when the cancer cells develop mutations or alternate signaling pathways that bypass the drug's mechanism of action, leading to treatment resistance. This can happen at various stages of treatment and may vary depending on the type of cancer being treated and the patient's individual response to the drug.

In some embodiments, a subject in need thereof may have a secondary cancer as a result of a previous therapy. “Secondary cancer” means cancer that arises due to or as a result from previous carcinogen therapies, such as chemotherapy.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in size of a tumor. A reduction in size of a tumor may be referred to as “tumor regression”. Preferably, after treatment, tumors size is reduced by 5% or greater relative to its size prior to treatment; more preferably, tumor size is reduced by 10% or greater; more preferably, reduced by 20 or greater; more preferably, reduced by 30% or greater; more preferably, reduced by 40 or greater, even more preferably, reduced by greater; and most preferably, reduced by greater than 75% or greater. Size of a tumor may be measured by any reproducible means of measurement. The size of a tumor may be measured as a diameter of the tumor.

Treating cancer results in decreased in number and size of tumors. Preferably, after treatment, tumor number or size is reduced by 5% or greater relative to umber prior to treatment; more preferably, tumor number or size is reduced by 10% or greater; more preferably, reduced by 20% or greater; more preferably, reduced by 30% or greater; more preferably, reduced by 40% or greater; even more preferably, reduced by 50% or greater; and most preferably reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 0% or greater; more preferably, reduced by 20% or greater; more preferably, reduced by 30% or greater; more preferably, reduce by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days, more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment at with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating fro a population the average length of survival following completion of a an act compound.

Treating cancer can result in increase in average survival time of a population of treated subjects in comparison to a population receiving monotherapy with a drug that is not a compound of the present invention, or a pharmaceutically acceptable salt or solvate thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt or solvate thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of related deaths per unit time following completion of a first round of treatment with active compound.

Treating cancer can result in a decrease in tumor growth rate. Preferably, after treatment, tumor growth rate is reduced by at least 5% relative to number prior to treatment; more preferably, tumor growth rate is reduced by a least 10%; more preferably, reduced by at least 20%; more preferably, reduced by at least 30%; more preferably, reduced by at least 40%, more preferably, reduced by at least 50%; even more preferably, reduced by at least 50%; and most preferably, reduced by at least 75%. Tumor growth rate may be measured by any reproducible me ent. Tumor growth rate can be measured according to a change in tumor diameter per unit time.

Treating cancer can result in a decrease in tumor regrowth. Preferably, after treatment, tumor growth is less than 5%; more preferably, tumor regrowth is less than 10%; more preferably, less than 20%; more preferably, less than 30%; more preferably, less than 40%; more preferably, less than 50% even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation Preferably, after treatment, the rate of cellular proliferation is reduced by at least 5%; more preferably, by at least 10%; more preferably, by at least 20%; more preferably, by at least 30%; more preferably by at least 40%; mort preferably, at least 50%; even more preferably, by at least 50%; and most preferably, by at least 75% The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area of zone of cellular proliferation is reduced by at least 5% relative to its size prior to treatment; more preferably, reduced by at least 10%; more preferably, reduced by at least 20%; more preferably, reduced by at least 30%; more preferably, reduced at least 40%; more preferably, reduced by at least 50%; even more preferably, reduced by at least 50%; and most preferably, reduce by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%, more preferably, reduced by at east 50%; even more preferably, reduced by at least 50%; and most preferably, reduced by at least 75%. An abnormal cellular appearance or morphology can be measured by any reproducible means of measurement. An abnormal cellular morphology can be measured by microscopy, e.g., using an inverted tissue culture microscope. An abnormal cellular morphology can take the form of nuclear pleiomorphism.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by ax y reproducible means. A number of cells in a population can be measured by fluorescence activated cell sorting (FACS), immunofluorescence microscopy and light microscopy. Methods of measuring cell death are as shown in Li et al., Proc. Natl. Acad Sci. USA. 100 (3): 2674-8, 2003. In an aspect, cell death occurs by apoptosis.

Preferably, an effective amount of a composition of the present invention, or a pharmaceutically acceptable salt or solvate thereof, is not significantly cytotoxic to normal cells. A therapeutically effective mount of a compound is not significantly cytotoxic to normal cells if administration of the compound in a therapeutically effective amount does not induce cell death in greater than 10% of normal calls. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a composition of the present invent or pharmaceutically acceptable salt or solvate thereof, can induce, or activate cell death actively in cancer cells. Administering to a subject in need thereof a compound of the present invention, or a pharmaceutically acceptable salt or solvate thereof, can induce or activate cell death selectively in cancer cells. Contacting a cell with a composition of the present invention, or a pharmaceutically acceptable salt or solvate thereof, can induce cell death selectively in one or more cells affected by a cell proliferative disorder. Preferably, administering to a subject in need thereof a composition of the present invention, or a pharmaceutically acceptable salt or solvate thereof, induces cell death selectively in one or more cells affected by a cell proliferative disorder.

The present invention relates to a method of treating or preventing cancer by administering a composition of the present invention, or a pharmaceutically acceptable salt or solvate thereof, to a subject in need thereof, where administration of the composition of the present invention, or a pharmaceutically acceptable salt or solvate thereof, results in one or more of the following: prevention of cancer cell proliferation by accumulation of cells in one or more phases of the cell cycle (e.g. G1, G1/S, G2/M), or induction of cell senescence, or promotion of tumor cell differentiation; promotion of cell death in cancer cells via cytotoxicity necrosis or apoptosis, without a significant amount of cell death in normal cells, antitumor activity in animals with a therapeutic index of at least 2. As used herein, “therapeutic index” is the maximum tolerated dose divided by the efficacious dose.

The term “kit” means a combination partner as defined above can be dosed Independently or by use of different fixed combinations with distinguished amounts of the combination partners, i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes.

One skilled in the art may refer to general reference texts for detailed descriptions of known techniques discussed herein or equivalent techniques. These texts can, of course, also be referred to in making of using an aspect of the invention.

LP-184 and LP-2 long to the acylfulvene compound family known to induce DNA lesions repaired by the Transcription-Coupled Nucleotide Excision Repair (TC-NER) pathway.

### EXAMPLES

The following non-limiting examples illustrate the methods of the present disclosure.

### Example 1

Triple-negative breast cancer (TNBC), a subtype of breast cancer that does not express estrogen receptor (ER), progesterone receptor (PR), or human epidermal growth factor receptor 2 (HER-2) is characterized by high invasiveness, high metastatic potential, high relapse rate and poor prognosis. Due to its special molecular phenotype, TNBC is not sensitive to endocrine therapy or targeted therapy. Therefore, chemotherapy is the main systemic treatment, but the efficacy of conventional postoperative adjuvant chemoradiotherapy is poor LP-184, an acylfulvene-derived prodrug, is activated by the oxidoreductase me PTGR1 and is synthetically lethal in tumors harboring DNA damage repair defects including Homologous Recombination (HR) deficiencies, for the treatment of solid tumor indications including TNBC

Cell viability assays were used to generate LP-184 IC50s in breast cancer cell lines in vitro. Subcutaneous patient derived TNBC xenograft mouse models were used to determine tumor volume responses in vivo. Xenograft tumors were derived from treatment-naive HR deficient primary TNBC patients with known BRCA1/2 LOH and who subsequently progressed on PARP inhibitor Olaparib.

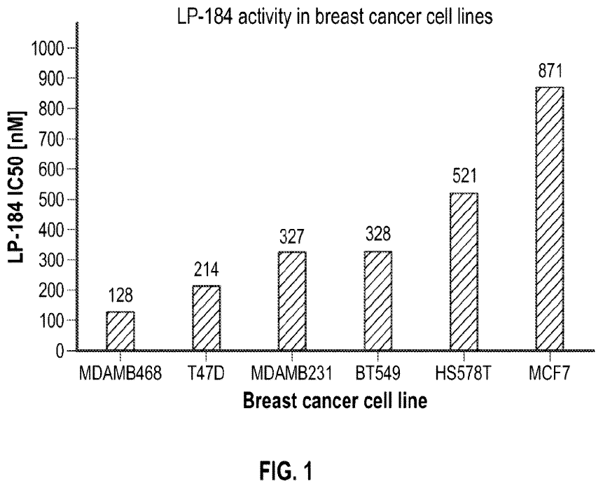

FIG. 1 shows that nanomolar concentrations of LP-184 inhibited in vitro viability of 6 NCI-60 breast cancer cell lines (median IC50=327 nM).

### Example 2—LP-184 Activity in Combination with a PARP Inhibitor in Triple Negative Breast Cancer

PDX models of TNBC have be n established by several groups wing a variety of methods, usually involving engraftment of frozen or fresh core needle biopsy-derived tumor fragments or dissociated tumor cells into mammary glands of immune-compromised mice. Such models are widely used to evaluate preclinical assessment of any new therapeutic approaches. The PDX model for this study was TNBC tumor (invasive ductal carcinoma) were engrafted in the flank of female NOD scid gamma (NSG) mice. Tumor size was measured every 7 days. LP-184 or hydroxyureamethylacylfulvene treatment resulted in complete tumor regression in a PDX model of TNBC that is HR deficient and resistant to PARP inhibitors and doxorubicin/cyclophosphamide.

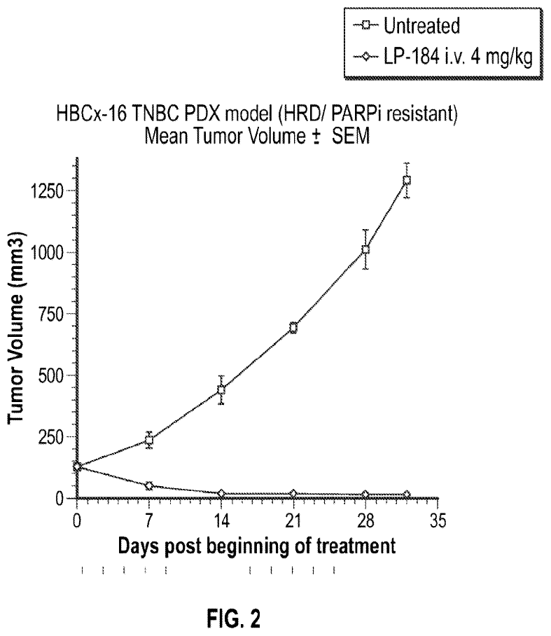

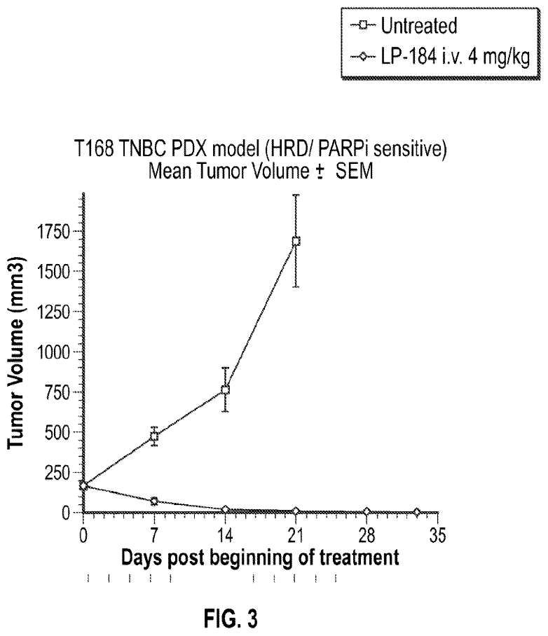

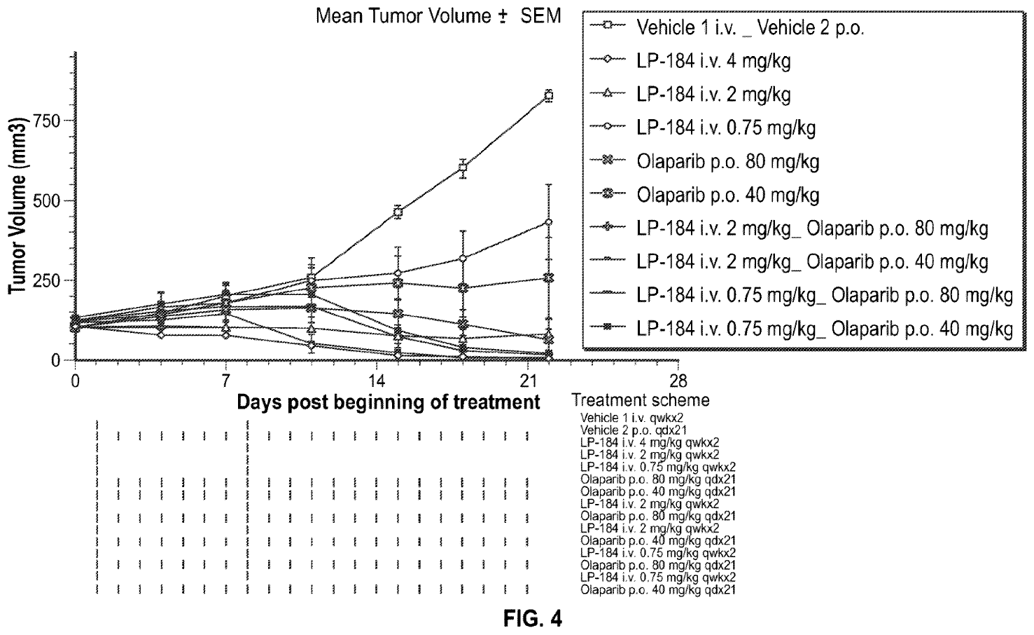

Patient-derived xenograft tumors of triple negative breast cancer were implanted subcutaneously in mice. Two PDX models of TNBC models, a PARPi sensitive model HBCx10 (BRCA2 mutant) and a PARPi resistant model HBCx28 (BRCA1 mutant), were treated with LP-184 and/or PARPi inhibitor Olaparib at different dose levels. Mice were randomly allocated per treatment arm with an established growing tumor between 60 and 200 mm³. Tumor volumes and mouse body weights were measured twice weekly. For HBCx10, LP-184 was dosed intravenously on Days 1 and 8 and Olaparib was dosed orally daily for 21 days. For HBCx28, LP-184 was dosed intravenously on Days 1, 4, 8, 11 and Olaparib was dosed orally daily for 21 days. In HBCx10, LP-184 treatment at 2 mg/kg (day 1 and 8 in a 21 day cycle) is comparable with 80 mg/kg daily Olaparib with a similar trend of tumor regression in each case. The combination of LP-184 (0.75 mg/kg)+Olaparib (40 mg/kg) showed highest synergy relative to individual treatments. Similarly, the combination of LP-184 (0.75 mg/kg)+Olaparib (80 mg kg) was synergistic. These results are displayed in FIGS. 2 and 3, which shows in TNBC in which HBCx10 tumor volume vs time during treatment with LP-184 and/or Olaparib was measured. FIG. 3 shows HBCx10 tumor volume in a PDX HBCx10 model over a treatment with LP-184 and/or Olaparib in HRD positive PARPi sensitive tumors. FIG. 4 shows tumor volume in a PDX HBCx10 model over a treatment with LP-184 and Olaparib.

At Day 22 percentage tumor growth inhibition (TGI %) values shown in Table 1 below demonstrate the synergy between LP-184 (0.75 mg/kg)+Olaparib (40 mg/kg).

TABLE 1
(HBC × 10 Day 22 tumor growth inhibition)

| | | | | | Day 22 |
|---|---|---|---|---|---|
| No | Agent 1 | Dose | Agent 2 | Dose | TGI % |
| 1 | Vehicle | — | Vehicle | — | — |
| | 1 | | 2 | | |
| 2 | LP-184 | 4 mg/kg | | | 113 |
| 3 | LP-184 | 2 mg/kg | | | 103 |
| 4 | LP-184 | 0.75 mg/kg | | | 66 |
| | | | Olaparib | 80 mg/kg | 105 |
| 6 | | | Olaparib | 40 mg/kg | 85 |
| 7 | LP-184 | 2 mg/kg | Olaparib | 80 mg/kg | ND |
| 8 | LP-184 | 2 mg/kg | Olaparib | 40 mg/kg | 113 |
| 9 | LP-184 | 0.75 mg/kg | Olaparib | 80 mg/kg | 112 |
| 10 | LP-184 | 0.75 mg/kg | Olaparib | 40 mg/kg | 112 |

In HBCx28, LP-184 treatment at 2 mg/kg (days 1, 4, 8, 11 or twice a week for 2 weeks) is more potent than any olaparib dose level. The combination of LP-184 (0.75 mg/kg)+Olaparib (80 mg/kg) was synergistic.

Further, at Day 21 percentage tumor growth inhibition (TGI %) values shown in Table 2 below demonstrate synergy between LP-184 (0.75 mg/kg)+Olaparib (80 mg/kg). LP-184 single agent at 2 and 4 mg/kg shows the highest tumor regression.

TABLE 2
HBC × 28 Day 21 tumor
growth inhibition

| | | | | | Day 21 |
|---|---|---|---|---|---|
| No. | Agent 1 | Dose | Agent 2 | Dose | TGI % |
| 1 | Vehicle | — | Vehicle | — | — |
| | 1 | | 2 | | |
| | LP-184 | 4 mg/kg | None | n/a | 17 |
| 3 | LP-184 | 2 mg/kg | None | n/a | 116 |
| 4 | LP-184 | 0.75 mg/kg | None | n/a | 45 |
| 5 | None | | Olaparib | 80 mg/kg | 12 |
| 6 | None | N/a | Olaparib | 40 mg/kg | 19 |
| 7 | LP-184 | 2 mg/kg | Olaparib | 80 mg/kg | 116 |
| 8 | LP-184 | 2 mg/kg | Olaparib | 40 mg/kg | 110 |
| 9 | LP-184 | 0.75 mg/kg | Olaparib | 80 mg/kg | 81 |
| 10 | LP-184 | 0.75 mg/kg | Olaparib | 40 mg/kg | 51 |

### Example 4—Synergy

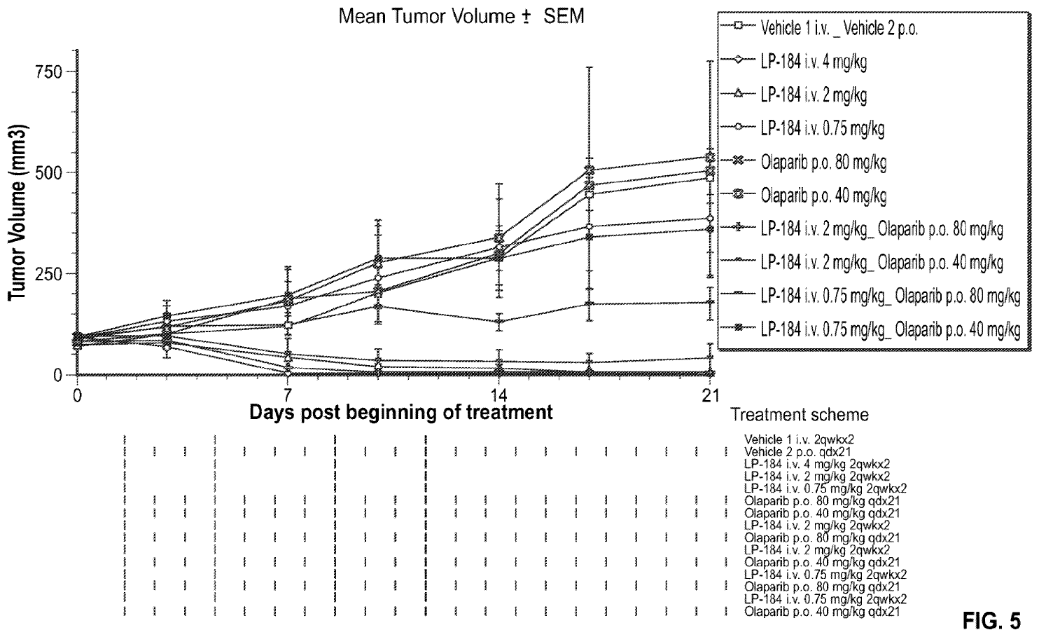

FIG. 4 and FIG. 5 shows that LP-184 and Olaparib illustrate synergy in TNBC HBCx10 and HBCx28 models, respectively. The tumor volume in HBCx10 in LP-184 treatments at 2 mg/kg (day 1 and 8 in a 21-day cycle) was comparable with 80 mg/kg daily Olaparib with a similar trend of tumor regression ach case. The combination of LP-184 (0.75 mg/kg)+Olaparib (40 mg/kg) showed highest synergy relative to individual treatments.

### Example 4—PARP Inhibitor Resistance

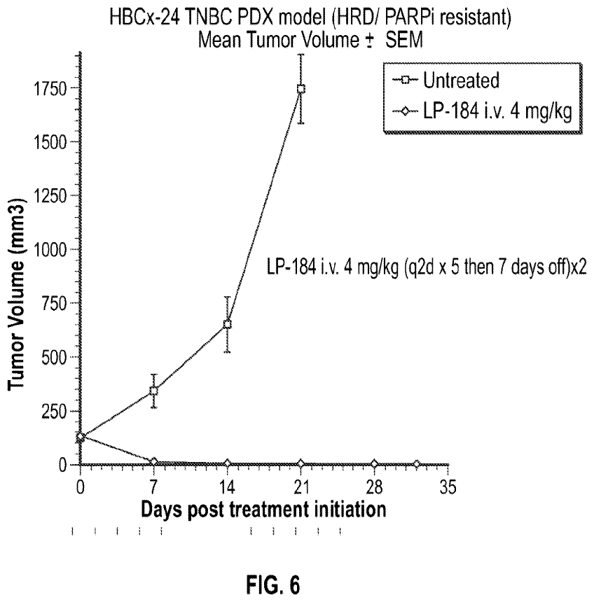

In the absence of any effective genetically engineered mouse model for TNBC, PDX models are widely used to evaluate preclinical assessment of any new therapeutic approaches. FIG. 6 shows complete tumor regression in an HR deficient, multi-drug resistant (PARP inhibitor resistant) TNBC in PDX HBCx-24 model. The PDX model for this study was TNBC tumor (invasive ductal carcinoma) were engrafted in the flank of female NOD scid gamma (NSG) mice. Tumor size was measured every 7 days. Complete tumor regression observed in an HR deficient, multi-drug resistant TNBC PDX model HBCx-24 with 4 mg/kg LP-184 i.v. treatment over 2 cycles; 107% TGI was at day 32 (FIG. 6).

1. A method of treating breast cancer that is PARP refractory or resistant in a patient in need thereof, the method comprises administering to a subject in need of treatment a combination comprising:
a. a therapeutically effective amount of an illudin or an illudin analog thereof, derivative, or a pharmaceutically acceptable salt thereof; and
b. a therapeutically effective amount of a PARP inhibitor or a pharmaceutically e salt thereof,
wherein the subject has relapsed cancer and/or refractory cancer; and the subject has been treated previously with the PARP inhibitor; and the subject is treated with the combination after the cancer is refractory and resistant to PARP inhibitor.

2. The method of claim 1, wherein the subject subsequently relapsed more than about 1 month following the cessation of treatment with the PARP inhibitor.

3. The method of claim 1, wherein the illudin analog is an acylfulvene.

4. The method of claim 1, wherein the illudin analog is HydroxyUreaMethylAcylfulvene.

5. The method of claim 1, wherein the illudin analog has the following structure:

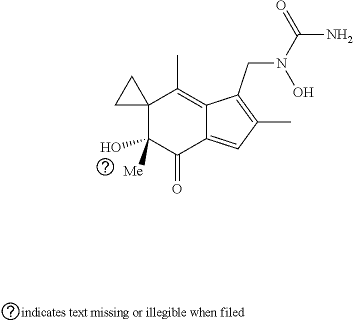 [SMILES: CC1=C(CN(O)C(N)=O)C2=C(C)C3(CC3)[C@@](C)(O)C(=O)C2=C1]

6. The method of claim 1, wherein the illudin analog has the following structure:

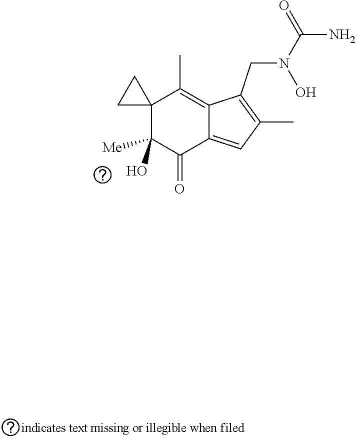 [SMILES: CC1=C(CN(O)C(N)=O)C2=C(C)C3(CC3)[C@](C)(O)C(=O)C2=C1]

7. The method of claim 1, wherein the illudin analog is Irofulven.

8. The method of claim 1, wherein the PARP inhibitor is administered after at least two rounds of the PARP inhibitor treatments.

9. The method of claim 1, wherein the PARP inhibitor or a pharmaceutically acceptable salt, solvate or hydrate thereof and the illudin or an illudin analog are for separate, simultaneous or sequential use or administration.

10. The method of claim 1, wherein the PARP inhibitor is administrated at a dose of 200 to 1200 mg daily.

11. The method of claim 1, wherein the illudin analog and the PARP inhibitor are administered separately.

12. The method of claim 1, wherein the illudin analog and the PARP inhibitor are administered daily.

13. The method of claim 1, wherein the illudin analog and the PARP inhibitor are administered sequentially.

14. The method of claim 1, wherein the active agents are administered as a co-formulation.

15. The method of claim 1, wherein an illudin or an analog thereof administration is before, during, or after the PARP inhibitor administration.

16. The method of claim 1, the method further comprising administering radiotherapy, chemotherapy to, performing surgery on, the subject before, during, or following the illudin and/or administering the PARP inhibitor.

17. The method of claim 1, wherein the one or more PARP inhibitor is selected from the group consisting of olaparib, rucaparib, and niraparib.

18. The method of claim 1, wherein the subject is an animal.

19. The method of claim 1, wherein the subject or mammal is a human.

20. The method of claim 5, further comprising subjecting the subject to radiation therapy before, after, or during treatment with HydroxyUreaMethylAcylfulvene.

21. The method of claim 1, further comprising administering an additional therapeutic agent selected from the group consisting of cisplatin, paclitaxel, and other available therapies.

22. The method of claim 1, wherein the breast cancer is triple negative breast cancer.

23. A method of treating breast cancer, the method comprises administering to a subject in need of treatment a combination comprising:
a. a therapeutically effective amount of an illudin or an illudin analog thereof, derivative, or a pharmaceutically salt thereof; and
b. a therapeutically effective amount of a PARP inhibitor or a pharmaceutically acceptable salt thereof;
wherein the breast cancer is triple negative breast cancer.

24. A kit for the treatment of cancer in a subject comprising a therapeutically effective amount of an illudin or an illudin analog thereof, derivative, or a pharmaceutically acceptable salt thereof, and a therapeutically effective amount of the PARP inhibitor or an analog, derivative, or a pharmaceutically acceptable salt thereof, wherein the subject has relapsed cancer and/or refractory cancer, and the subject has been treated previously with the Ibrutinib; and the subject is treated with the combination after the cancer is refractory and resistant to Ibrutinib.

25. The pharmaceutical composition claim 24, wherein the illudin analog is HydroxyUreaMethylAcylfulvene.

26. The pharmaceutical composition of claim 24, wherein the illudin analog has the following structure:

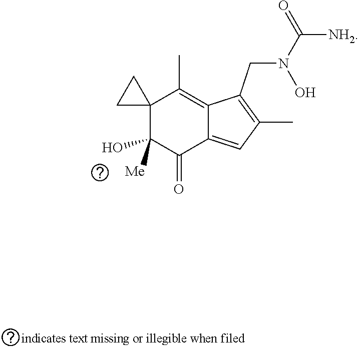 [SMILES: CC1=C(CN(O)C(N)=O)C2=C(C)C3(CC3)[C@@](C)(O)C(=O)C2=C1]

27. The pharmaceutical composition of claim 30, wherein the illudin analog has the following structure: 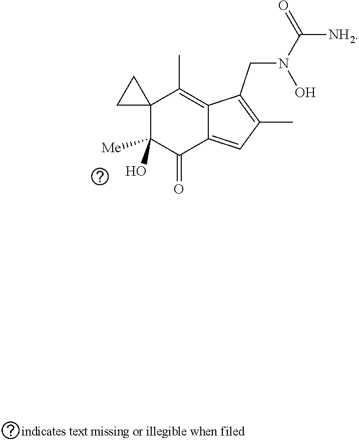 [SMILES: CC1=C(CN(O)C(N)=O)C2=C(C)C3(CC3)[C@](C)(O)C(=O)C2=C1]
